(19) United States
(12) Patent Application Publication
(10) Pub. No.: US 2024/0358657 A1
(43) Pub. Date: Oct. 31, 2024

(54) erecting/erection ,treat impotence.

(71) Applicant: Menni Menashe Zinger, Ashkelon (IL)

(72) Inventor: Menni Menashe Zinger, Ashkelon (IL)

(21) Appl. No.: 18/140,644

(22) Filed: Apr. 28, 2023

(51) Int. Cl. A61K 31/135; A61K 36/898

(52) U.S. Cl. CPC A61K 31/135; A61K 36/898

(57) ABSTRACT

At this invention I present ways to improve erecting/erections and treat impotence.

By a test I did the syrup for relief at coughing might cause erecting/erection treat impotence even without thinking on sex, at time of day sleep for short time as 20 minutes at man at his late 40^{th} who is talking several medication for several medical illnesses/problems and has a past of surgeries at his pines by talking 5 milligrams of the syrup before that sleep.

1. Using Syrup for relief at coughing, ammonium chloride diphenhydramine hci, in order to improve erecting erection.

2. using vanilla as medication for improving erecting erection, treat impotence.

3. using tchina and mayonnaise and eggplant or humus food types/salads ingredients in order to increase pines significantly at un erected state and erection state too.
